(19) United States
(12) Patent Application Publication
(10) Pub. No.: US 2025/0025342 A1
(43) Pub. Date: Jan. 23, 2025

(54) METHOD FOR PROVIDING CONTROL DATA FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/776,688

(22) Filed: Jul. 18, 2024

(30) Foreign Application Priority Data
Jul. 21, 2023 (DE) 10 2023 119 297.3

(51) Int. Cl. A61F 9/008

(52) U.S. Cl. CPC A61F 9/00804; A61F 2009/00848; A61F 2009/00872

(57) ABSTRACT

The invention relates to a method for providing control data for an ophthalmological laser (12) of a treatment apparatus (10). By a control device (18), determining (S10) corneal parameters of an anterior surface (24) of a cornea (16) from predetermined examination data; determining (S12) an epithelial map of an epithelial layer (26) of the cornea (16) from the predetermined examination data, wherein a thickness of the epithelial layer (26) is provided in the epithelial map; calculating (S14) a stromal wavefront map depending on the determined corneal parameters of the anterior surface (24) of the cornea and the determined epithelial map; determining (S16) correction data for correcting a visual disorder based on the stromal wavefront map; and providing (S18) the control data, which includes the correction data determined based on the stromal wavefront map are effected.

### FIELD

The invention relates to a method for providing control data for an ophthalmological laser of a treatment apparatus. Furthermore, the invention relates to a control device, which is formed to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device may for example be formed such that laser pulses effect a photodisruption and/or ablation in a focus area situated within the organic tissue to remove a tissue, in particular a tissue lenticule, from the cornea.

A treatment method for correcting a visual disorder is the transepithelial photorefractive keratectomy (Trans-PRK), in which corneal layers, in particular also an epithelial layer, are ablated from the surface; but the epithelial layer can regrow. Accordingly, only the tissue removal, which is performed in a stroma of the cornea, is persistent. Therefore, the additional ablation of the epithelial layer is to be taken into account in the treatment planning, wherein only a standardized epithelial layer with a constant thickness is planned up to now, and in particular a patient-individual shape of the epithelial layer, which may have an irregular epithelial shape, has been left out of consideration up to now.

### SUMMARY

It is the object of the invention to provide control data for a treatment with an ophthalmological laser, by which an improved correction result of a cornea may be achieved.

This object is solved by the independent claims. Advantageous embodiments of the invention are disclosed in the dependent claims, the present description as well as the Figs.

The invention is based on the idea that one determines a stromal component of the visual disorder separately from a corneal surface in that the portion of the epithelial layer is subtracted. Thus, the treatment planning may be directly performed for the stroma, which does not regrow, and thus the treatment planning may be planned only for that area, in which the correction remains after the treatment.

An aspect of the invention relates to a method for providing control data for an ophthalmological laser of a treatment apparatus, wherein the method comprises the following steps performed a control device. Therein, an appliance or an appliance component, in particular a computer or processor, which may automatically or semi-automatically perform the following steps, is to be understood by a control device.

In the method, determining corneal parameters of an anterior surface of a cornea from predetermined examination data, determining an epithelial map of an epithelial layer of the cornea from the predetermined examination data, wherein a thickness of the epithelial layer is provided in the epithelial map, calculating a stromal wavefront map depending on the determined corneal parameters of the anterior surface of the cornea and of the determined epithelial map, determining correction data for correcting a visual disorder based on the stromal wavefront map and providing the control data, which includes the correction data determined based on the stromal wavefront map, are effected.

In other words, examination data of an eye, in particular a cornea of the eye, may be predetermined, from which corneal parameters of the anterior surface may be ascertained. For example, topography measurements and/or wavefront measurements may be performed, and the corneal parameters may, for example, include an anterior corneal wavefront map and/or an anterior corneal surface map. A wavefront error of the eye or the cornea is meant by a wavefront or wavefront map, which indicates a wavefront deformation as a function of the location. An anterior corneal surface map may, for example, include height data or profile data of the anterior surface of the cornea. It may, for example, be ascertained by a topography measurement. Furthermore, an epithelial map of an epithelial layer may be determined from the examination data, wherein the epithelial map may indicate a thickness of the epithelial layer depending on the location in the cornea.

With the aid of the corneal parameters and the epithelial map, a stromal wavefront map may then be determined, which provides the wavefront deformation by the stroma. In particular, effects of the epithelial layer, which may be ascertained from the epithelial map, may be subtracted from the corneal parameters, which provide the characteristics of the entire cornea, to obtain the stromal wavefront map from the difference.

By the stromal wavefront map, the correction may then be planned, which may be provided as correction data. This means, it may be ascertained, which changes have to be performed on the stroma to, for example, correct a visual disorder. These corrections may then be provided in the form of control data, which may be used for controlling the treatment apparatus and/or the laser. The control data may include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser may be included in the control data.

By the invention, the advantage arises that changes may be planned only on tissue of the cornea, in particular the stroma, which permanently remain, since the epithelial layer regrows and thus changes on the epithelial layer do not persistently remain. Furthermore, it is thus possible to consider effects of irregular epithelial layers since a simplified or averaged model does not have to be assumed for the epithelial layer. Thus, the correction of visual disorders may overall be improved, in particular for a Trans-PRK.

The invention also includes embodiments, by which additional advantages arise.

In an embodiment, the epithelial map indicates the thickness of the epithelial layer in a z-direction for respective [x,y] positions. In other words, thus, one obtains a map in cartesian coordinates, in which the respective thickness values or height values of the epithelial layer for respective [x,y] positions, in particular for each [x,y] position, of the cornea are indicated on the z-axis or in the direction of an optical axis of the cornea and parallel to each other. Therein, the x,y plane is the plane in top view to the eye/the cornea. In particular, an [r,phi] position may also be indicated for the map instead of an [x,y] position, thus, the map may be provided in cylindrical coordinates.

In a further embodiment, an anterior corneal surface map is provided by the corneal parameters, which includes height data of the anterior surface of the cornea, wherein an anterior stromal surface map is determined by subtracting the epithelial map from the anterior corneal surface map, and the stromal wavefront map is determined from the anterior stromal surface map. In other words, the corneal parameters may include at least an anterior corneal surface map, in which a height profile or height data of the anterior surface of the cornea is present. Alternatively or additionally, a corneal wavefront map of the anterior surface of the cornea may also be provided as corneal parameters, from which height data of the anterior surface of the cornea may be ascertained. By the anterior corneal surface map, a difference to the epithelial map may then be formed such that a stromal surface map is obtained. In other words, the profile of the epithelial layer may be subtracted from the height profile of the surface of the cornea to obtain the profile of the stroma. The profile of the stroma in the form of the anterior stromal surface map may then be converted to a stromal wavefront map, in particular with the formula:

$$stromaWF ( x , y ) = ( stromaTH ( x , y ) - avg ( Stroma ) ) * ( n\_Stroma - 1 ) ,$$

wherein stromaWF(x,y) represents the stromal wavefront map, stromaTH(x,y) the anterior stromal surface map, avg(Stroma) an average height value (average value) of the stroma and n_Stroma a refractive index of the stroma. In other words, the topology of the stroma may be determined from the topology of the corneal surface and the topology of the epithelial layer, wherein a wavefront for treatment planning may be ascertained by the topology of the stroma.

In a further embodiment, an anterior corneal wavefront map is provided by the corneal parameters, wherein an epithelial wavefront map is calculated from the epithelial map for calculating the stromal wavefront map, wherein the stromal wavefront map is determined by subtraction of the epithelial wavefront map from the anterior corneal wavefront map. In other words, a wavefront measurement of the cornea, which provides the anterior corneal wavefront map, may be calculated by subtraction with an epithelial wavefront map. Herein, the epithelial wavefront map may be obtained by a calculation from the epithelial map, thus a conversion of a topology to a wavefront, in particular with the formula:

$$epiWF ( x , y ) = ( epiTh ( x , y ) - avg ( epi ) ) * ( n\_epi - 1 ) ,$$

wherein epiWF(x,y) represents the epithelial wavefront map, epiTH(x,y) the epithelial map, avg(epi) an average thickness value (average value) of the epithelial layer and n_epi a refractive index of the epithelium. In other words, the wavefront of the stroma may be determined from the wavefront of the corneal surface and the wavefront of the epithelial layer, wherein the wavefront of the epithelial layer may be ascertained by the topology of the epithelial layer. By this embodiment, the advantage arises that compared to the previously mentioned variant, in which the stromal wavefront map has been calculated based on the topologies, a lower error propagation arises, which improves a determination.

In a further embodiment, an adapted tissue removal on the stroma is planned for the correction data, by which a change of the epithelial layer after an epithelial recovery is compensated for. In other words, the epithelial layer can again regrow after the treatment, wherein it does not take the original shape due to the treatment, but, for example, has irregularities, which can generate visual disorders, in particular higher order aberrations. This means that the epithelial layer is not identically recovered anymore as before the treatment, but has differences, wherein these differences may be estimated and compensated for. This changed epithelial layer may also be taken into account in the treatment planning on the stroma in that a change of the epithelial layer is planned by an adapted tissue removal in the stroma. This means that additional tissue may be removed from the stroma, which may compensate for an effect of the changed epithelial layer. For estimating the change of the epithelial layer after the epithelial recovery, it can for example be assumed that the preoperative epithelial layer is equal to the postoperative epithelial layer after the epithelial recovery, but has a different curvature due to the tissue removal on the stroma. A further alternative thereto is in that one assumes an average epithelial layer shape for the postoperative epithelial layer from statistics of a predetermined population, by which changes of the epithelial layer after the treatment are provided. An average value of the average epithelial layer and the preoperative epithelial layer may for example also be formed to model a postoperative epithelial layer. By these embodiments, the advantage arises that additional impairing effects may be corrected in improved manner.

In a further embodiment, different laser interactions with the tissue of the epithelial layer and the stroma are taken into account in the control data. In other words, epithelial layer tissue and stroma tissue may differently respond to radiated laser pulses. For example, different energy densities may be required for separating the respective tissues, wherein they may be taken into account in the control data. Thus, a laser pulse energy depending on the tissues and/or a number of laser pulses per surface or volume unit may for example be adapted. Hereby, the advantage arises that a treatment of the cornea may be improved.

A further possibility provides that the epithelial map is determined by optical coherence tomography and/or ultrasound, in particular ultrahigh frequency ultrasound (UHF ultrasound). In other words, the thickness of the epithelial layer may be directly measured to provide the epithelial map. Herein, in particular the optical coherence tomography and/or an ultrasound of the cornea are suitable.

A further aspect relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect of the invention relates to a control device, which is formed to perform the steps of at least one embodiment of one or both of the previously described methods. Thereto, the control device may comprise a computing unit for electronic data processing such as, for example, a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser may be formed to at least partially separate a predefined corneal volume with predefined interfaces of a human or animal eye by optical breakdown, in particular at least partially separate it by photodisruption and/or to ablate corneal layers by (photo)ablation and/or to effect a laser-induced refractive index change in the cornea and/or the eye lens and/or to increase a crosslinking of the cornea.

In a further embodiment of the treatment apparatus according to the invention, the laser may be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown may be spatially narrowly limited such that a high incision accuracy is allowed in the generation of the interfaces. In particular, the range between 700 nm and 780 nm may also be selected as the wavelength range.

In a further embodiment of the treatment apparatus according to the invention, the control device may comprise at least one storage device for at least temporary storage of at least one control dataset, wherein the control dataset or datasets include(s) control data for positioning and/or for focusing individual laser pulses in the cornea; and may comprise at least one beam device for beam guidance and/or beam shaping and/or beam deflection and/or beam focusing of a laser beam of the laser.

A further aspect of the invention relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster may access the computer-readable medium and read out the content thereof. The storage medium is, for example, formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the embodiments of another one of the aspects of the invention. Thus, the features of the embodiments of the invention may be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, configurations are also to be regarded as encompassed and disclosed by the invention, which are not explicitly shown or explained in the figures, but arise from and may be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematically illustrated structure of corneal layers of a cornea.

FIG. 3 depicts a schematic method diagram for providing control data according to an exemplary embodiment.

In the figures, identical or functionally identical elements are provided with the same reference characters.

### DETAILED DESCRIPTION

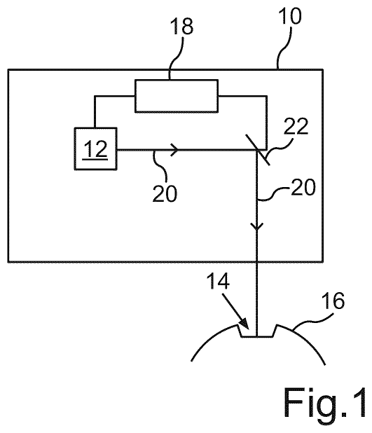

FIG. 1 shows a schematic representation of a treatment apparatus 10 with an ophthalmological laser 12 for removing a tissue 14 from a human or animal cornea 16 by photodisruption and/or ablation. For example, the tissue 14 may be separated by the eye surgical laser 12 by ablating corneal layers from the cornea 16 for correcting a visual disorder. A correction profile or a geometry of the tissue 14 to be removed may be set by correction data, which may be ascertained by a control device 18. In particular, control data may be provided, which includes correction data, wherein the control device 18 may control the laser 12 and/or the treatment apparatus 10 by the control data such that pulsed laser pulses are emitted in a pattern predefined by the control data into the cornea 16 of the eye to remove the tissue 14. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22 such as for example a rotation scanner, to remove the tissue 14. The beam deflection device 22 may also be controlled by the control device 18 to remove the tissue 14.

In addition, the control device 18 optionally comprises a storage device (not illustrated) for at least temporary storage of at least one control dataset, wherein the control dataset or datasets include(s) control data for positioning and/or for focusing individual laser pulses in the cornea.

Usually, a treatment planning for ascertaining the correction data for removing the tissue 14 is based on a corneal wavefront map. However, it is disadvantageous herein that effects of the epithelial layer are also present in the corneal wavefront map, which cannot be compensated for by the treatment since the epithelial layer regrows. In order to perform an improved treatment, in particular in a trans-photorefractive keratectomy, it may be provided that a treatment planning is not performed on the corneal wavefront map, but directly on a stromal wavefront map to exclude effects of the epithelial layer in the treatment planning.

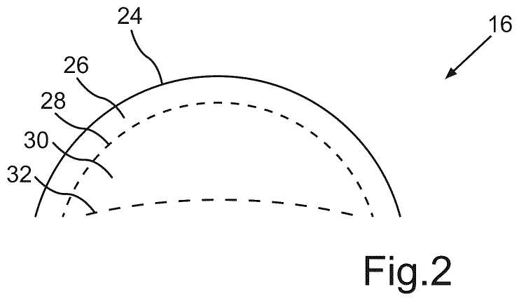

In FIG. 2, a schematic and simplified structure of a cornea 16 is represented, by which the layers of the cornea are illustrated. An anterior surface 24 forms the surface of the cornea 16. Herein, curvatures and elevations may for example be provided in the form of an anterior corneal surface map and/or a wavefront deformation on the anterior surface 24 may be provided in the form of an anterior corneal wavefront map.

Viewed in depth direction, from the direction of the corneal surface 24, the epithelial layer 26 follows. The epithelial layer or the epithelium includes epithelial cells and is about 40 to 60 μm thick, wherein the epithelial layer 26 can regrow after damage and/or removal. By measurements, in particular by an optical coherence tomography and/or by ultrasound, a thickness of the epithelial layer 26 may be determined, which can for example be provided in the form of an epithelial map.

The epithelial layer 26 is bounded by the Bowman's membrane 28, which delimits the epithelial layer 26 from the stroma 30. By the stroma 30, the main portion of the corneal thickness is provided, wherein the stroma 30 is about 400 to 500 μm thick. In contrast to the epithelial layer 26, the stroma 30 cannot regrow. Therefore, corrections of visual disorders by an ophthalmological laser may be provided in the stroma 30, whereby a persistent change of optical characteristics of the cornea 16 is provided. In particular, the stroma 30 may have other laser interaction characteristics than the epithelial layer 26 due to the tissue structure, wherefore optimized or different laser parameters may be planned for the respective layer 26, 30. Finally, the stroma 30 is terminated by the Descemet membrane 32. Further membranes and/or layers of the cornea are not illustrated in FIG. 2 for reasons of clarity.

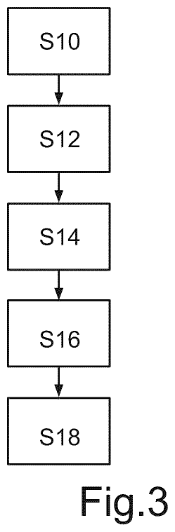

For an improved treatment planning, in particular in Trans-PRK treatments, the control device 18 may perform the method shown in FIG. 3.

In FIG. 3, a schematic method diagram for providing control data for an ophthalmological laser 12 of a treatment apparatus 10 is illustrated, wherein the method may be performed by the control device 18.

In a step S10, corneal parameters of an anterior surface 24 of a cornea 16 may be ascertained from predetermined examination data. For example, a topography measurement may be present, by which an anterior corneal surface map may be provided, and/or wavefront measurements may be performed, by which an anterior corneal wavefront map may be provided.

In a step S12, an epithelial map of an epithelial layer 26 of the cornea 16 may be ascertained from the predetermined examination data, wherein the epithelial map may indicate the thickness of the epithelial layer 26 in z-direction for respective [x,y] positions. In particular, the epithelial map may be determined by optical coherence tomography and/or ultrasound.

In a step S14, a stromal wavefront map may be calculated from the corneal parameters and the epithelial map. Therein, the stromal wavefront map indicates the wavefront deformation, which is generated exclusively due to the stroma 30. In order to calculate the stromal wavefront map, a difference of the respective topographies may be performed on the one hand or a difference of wavefronts on the other hand.

In the difference of the topographies, it may be provided that the corneal parameters include an anterior surface map, which comprises height data or elevations of the anterior surface 24 of the cornea 16. The epithelial map, which is also present as a topography, may be subtracted from the anterior stromal surface map, such that the surface of the stroma 30 is modeled, which may be provided as an anterior stromal surface map. The anterior stromal surface map may in turn be converted to a stromal wavefront map, by which correction data for correcting a visual disorder may be determined in a step S16, for example refraction data for spherical, cylindrical and/or astigmatism correction.

In the wavefront-based ascertainment of the stromal wavefront map, the corneal parameters may include an anterior corneal wavefront map of the corneal surface 24. The epithelial map of the epithelial layer 26 may be converted to a wavefront, such that an epithelial wavefront map is provided. Thus, a wavefront of the stroma 30 may be provided by difference of the epithelial wavefront map with the anterior corneal wavefront map, wherein this stromal wavefront map may then be provided for determining the correction data in a step S16.

Finally, in a step S18, control data for controlling the ophthalmological laser 12 and/or the treatment apparatus 10 may be provided, by which laser pulses for removing the tissue 14 may be radiated into the cornea 16 according to the correction data for correcting the visual disorder.

In particular, an adapted tissue removal for removing the tissue 14 in the stroma 30 may also be taken into account in the correction data, by which effects are compensated for, which occur by a changed epithelial recovery. Thus, a curvature of the epithelial layer before and after the treatment is, for example, different due to the tissue removal, wherein the changed curvature may be estimated in advance. Thus, effects such as, for example, aberrations, which arise by the changed curvature of the epithelial layer 26, may be planned in advance and be considered by the adapted tissue removal.

Overall, the examples show how a shape of the stroma 30 may be virtually determined and be used for treatment planning.

1. A method for providing control data for an ophthalmological laser of a treatment apparatus, wherein the method comprises the following steps performed by a control device:
determining corneal parameters of an anterior surface of a cornea from predetermined examination data;
determining an epithelial map of an epithelial layer of the cornea from the predetermined examination data, wherein a thickness of the epithelial layer is provided in the epithelial map;
calculating a stromal wavefront map depending on the determined corneal parameters of the anterior surface of the cornea and the determined epithelial map;
determining correction data for correcting a visual disorder based on the stromal wavefront map;
providing the control data, which includes the correction data determined based on the stromal wavefront map.

2. The method according to claim 1, wherein the epithelial map indicates the thickness of the epithelial layer in a z-direction for respective [x,y] positions.

3. The method according to claim 1, wherein an anterior corneal surface map is provided by the corneal parameters, which includes height data of the anterior surface of the cornea, wherein an anterior stromal surface map is determined by subtracting the epithelial map from the anterior corneal surface map, and the stromal wavefront map is determined from the anterior stromal surface map.

4. The method according to claim 1, wherein an anterior corneal wavefront map is provided by the corneal parameters, wherein an epithelial wavefront map is calculated from the epithelial map for calculating the stromal wavefront map, wherein the stromal wavefront map is determined by subtraction of the epithelial wavefront map from the anterior corneal wavefront map.

5. The method according to claim 1, wherein the correction data includes an adapted tissue removal in a stroma of the cornea as compensation for a change of the epithelial layer after an epithelial recovery.

6. The method according to claim 1, wherein different laser interactions with tissue of the epithelial layer and a stroma of the cornea are taken into account in the control data.

7. The method according to claim 1, wherein the epithelial map is determined by optical coherence tomography and/or ultrasound.

8. A control device, which is configured to perform the method according to claim 1.

9. A treatment apparatus with at least one eye surgical laser for removing a tissue from a cornea of a human or animal eye by optical breakdown, in particular by photodisruption and/or photoablation, and at least one control device according to claim 8.

10. (canceled)

11. A computer-readable medium for storing a computer program, the computer program comprising commands which cause a treatment apparatus to execute the method according to claim 1.
